(19) United States
(12) Patent Application Publication
(10) Pub. No.: US 2019/0378996 A1
(43) Pub. Date: Dec. 12, 2019

(54) MATERIALS FOR ORGANIC ELECTROLUMINESCENT DEVICES

(71) Applicant: Merck Patent GmbH, Darmstadt (DE)

(72) Inventors: Philipp STOESSEL, Frankfurt am Main (DE); Amir PARHAM, Frankfurt am Main (DE); Dominik JOOSTEN, Frankfurt am Main (DE); Aurélie LUDEMANN, Frankfurt am Main (DE); Tobias GROSSMANN, Darmstadt (DE); Jonas KROEBER, Frankfurt am Main (DE)

(21) Appl. No.: 16/463,586

(22) PCT Filed: Dec. 4, 2017
(86) PCT No.: PCT/EP2017/081288

§ 371 Date: May 23, 2019

(30) Foreign Application Priority Data
Dec. 5, 2016 (EP) 16202121.6

(51) Int. Cl. H01L 51/00; C07D 471/06; C09K 11/06; C07D 519/00; C07D 495/06; C07D 491/052

(52) U.S. Cl. CPC H01L 51/0072; C07D 471/06; C09K 11/06; H01L 51/0067; H01L 51/0073; H01L 51/0058; H01L 51/5016; H01L 51/0071; H01L 51/0054; H01L 51/0056; C07D 495/06; C07D 491/052; C07D 519/00

(57) ABSTRACT

The present invention relates to compounds suitable for use in electronic devices, and to electronic devices, especially organic electroluminescent devices, comprising these compounds.

The present invention relates to materials for use in electronic devices, especially in organic electroluminescent devices, and to electronic devices, especially organic electroluminescent devices comprising these materials.

Emitting materials used in organic electroluminescent devices (OLEDs) are frequently organometallic complexes which exhibit phosphorescence rather than fluorescence. For quantum-mechanical reasons, up to four times the energy efficiency and power efficiency is possible using organometallic compounds as phosphorescent emitters. In general terms, however, there is still a need for improvement in OLEDs, especially also in OLEDs which exhibit triplet emission (phosphorescence), for example with regard to efficiency, operating voltage and lifetime. The properties of phosphorescent OLEDs are not just determined by the triplet emitters used. More particularly, the other materials used, such as matrix materials, are also of particular significance here. Improvements to these materials and the charge transport properties thereof can thus also lead to distinct improvements in the OLED properties.

It is an object of the present invention to provide compounds suitable for use in an OLED, especially as matrix material for phosphorescent emitters. A further problem addressed by the present invention is that of providing further organic semiconductors for organic electroluminescent devices, in order thus to enable the person skilled in the art to have a greater possible choice of materials for the production of OLEDs.

It has been found that, surprisingly, particular compounds described below achieve this object and are of good suitability for use in OLEDs and lead to improvements in the organic electroluminescent device. These improvements relate particularly to the lifetime, efficiency and/or operating voltage. The present invention therefore provides these compounds and electronic devices, especially organic electroluminescent devices, comprising such compounds.

The present invention therefore provides a compound of formula (1)

where the symbols used are as follows:

X is the same or different at each instance and is CR or N;

Y is NR, BR′, O or S;

R is the same or different at each instance and is H, D, F, Cl, Br, I, NAr₂, N(R¹)₂, CN, NO₂, OR¹, SR¹, COOR¹, C(═O)N(R¹)₂, Si(R¹)₃, B(OR¹)₂, C(═O)R¹, P(═O)(R¹)₂, S(═O)R¹, S(═O)₂R¹, OSO₂R¹, a straight-chain alkyl group having 1 to 20 carbon atoms or an alkenyl or alkynyl group having 2 to 20 carbon atoms or a branched or cyclic alkyl group having 3 to 20 carbon atoms, where the alkyl, alkenyl or alkynyl group may in each case be substituted by one or more R¹ radicals and where one or more nonadjacent CH₂ groups may be replaced by Si(R¹)₂, C═O, NR¹, O, S or CONR¹, or an aromatic or heteroaromatic ring system which has 5 to 60 aromatic ring atoms, preferably 5 to 40 aromatic ring atoms, and may be substituted in each case by one or more R¹ radicals; at the same time, two R radicals together may also form an aliphatic or heteroaliphatic ring system;

R′ is an aromatic or heteroaromatic ring system which has 5 to 40 aromatic ring atoms and may be substituted by one or more R¹ radicals;

Ar is the same or different at each instance and is an aromatic or heteroaromatic ring system which has 5 to 40 aromatic ring atoms and may be substituted by one or more R¹ radicals;

R¹ is the same or different at each instance and is H, D, F, Cl, Br, I, N(R²)₂, CN, NO₂, OR², SR², Si(R²)₃, B(OR²)₂, C(═O)R², P(═O)(R²)₂, S(═O)R², S(═O)₂R², OSO₂R², a straight-chain alkyl group having 1 to 20 carbon atoms or an alkenyl or alkynyl group having 2 to 20 carbon atoms or a branched or cyclic alkyl group having 3 to 20 carbon atoms, where the alkyl, alkenyl or alkynyl group may in each case be substituted by one or more R² radicals and where one or more nonadjacent CH₂ groups may be replaced by Si(R²)₂, C═O, NR², O, S or CONR², or an aromatic or heteroaromatic ring system which has 5 to 40 aromatic ring atoms and may be substituted in each case by one or more R² radicals; at the same time, two or more R¹ radicals together may form a ring system;

R² is the same or different at each instance and is H, D, F or an aliphatic, aromatic or heteroaromatic organic radical, especially a hydrocarbyl radical, having 1 to 20 carbon atoms, in which one or more hydrogen atoms may also be replaced by F;

with the proviso that the compound of the formula (1) contains at least one substituent R′ and/or that the compound of the formula (1) contains at least one substituent R selected from the group consisting of NAr₂ and an aromatic or heteroaromatic ring system which has 5 to 60 aromatic ring atoms and may be substituted by one or more R¹ radicals.

An aryl group in the context of this invention contains 6 to 40 carbon atoms; a heteroaryl group in the context of this invention contains 2 to 40 carbon atoms and at least one heteroatom, with the proviso that the sum total of carbon atoms and heteroatoms is at least 5. The heteroatoms are preferably selected from N, O and/or S. An aryl group or heteroaryl group is understood here to mean either a simple aromatic cycle, i.e. benzene, or a simple heteroaromatic cycle, for example pyridine, pyrimidine, thiophene, etc., or a fused (annelated) aryl or heteroaryl group, for example naphthalene, anthracene, phenanthrene, quinoline, isoquinoline, etc. Aromatic systems joined to one another by a single bond, for example biphenyl, by contrast, are not referred to as an aryl or heteroaryl group but as an aromatic ring system.

An aromatic ring system in the context of this invention contains 6 to 60 carbon atoms in the ring system. A heteroaromatic ring system in the context of this invention contains 2 to 60 carbon atoms and at least one heteroatom in the ring system, with the proviso that the sum total of carbon atoms and heteroatoms is at least 5. The heteroatoms are preferably selected from N, O and/or S. An aromatic or heteroaromatic ring system in the context of this invention shall be understood to mean a system which does not necessarily contain only aryl or heteroaryl groups, but in which it is also possible for two or more aryl or heteroaryl groups to be joined by a nonaromatic unit, for example a carbon, nitrogen or oxygen atom. For example, systems such as fluorene, 9,9′-spirobifluorene, 9,9-diarylfluorene, triarylamine, diaryl ethers, stilbene, etc. shall also be regarded as aromatic ring systems in the context of this invention, and likewise systems in which two or more aryl groups are joined, for example, by a short alkyl group.

In the context of the present invention, an aliphatic hydrocarbyl radical or an alkyl group or an alkenyl or alkynyl group which may contain 1 to 40 carbon atoms and in which individual hydrogen atoms or CH₂ groups may also be substituted by the abovementioned groups is preferably understood to mean the methyl, ethyl, n-propyl, i-propyl, n-butyl, i-butyl, s-butyl, t-butyl, 2-methylbutyl, n-pentyl, s-pentyl, neopentyl, cyclopentyl, n-hexyl, neohexyl, cyclohexyl, n-heptyl, cycloheptyl, n-octyl, cyclooctyl, 2-ethylhexyl, trifluoromethyl, pentafluoroethyl, 2,2,2-trifluoroethyl, ethenyl, propenyl, butenyl, pentenyl, cyclopentenyl, hexenyl, cyclohexenyl, heptenyl, cycloheptenyl, octenyl, cyclooctenyl, ethynyl, propynyl, butynyl, pentynyl, hexynyl, heptynyl or octynyl radicals. An alkoxy group OR¹ having 1 to 40 carbon atoms is preferably understood to mean methoxy, trifluoromethoxy, ethoxy, n-propoxy, i-propoxy, n-butoxy, i-butoxy, s-butoxy, t-butoxy, n-pentoxy, s-pentoxy, 2-methylbutoxy, n-hexoxy, cyclohexyloxy, n-heptoxy, cycloheptyloxy, n-octyloxy, cyclooctyloxy, 2-ethylhexyloxy, pentafluoroethoxy and 2,2,2-trifluoroethoxy. A thioalkyl group SR¹ having 1 to 40 carbon atoms is understood to mean especially methylthio, ethylthio, n-propylthio, i-propylthio, n-butylthio, i-butylthio, s-butylthio, t-butylthio, n-pentylthio, s-pentylthio, n-hexylthio, cyclohexylthio, n-heptylthio, cycloheptylthio, n-octylthio, cyclooctylthio, 2-ethylhexylthio, trifluoromethylthio, pentafluoroethylthio, 2,2,2-trifluoroethylthia, ethenylthio, propenylthio, butenylthio, pentenylthio, cyclopentenylthio, hexenylthio, cyclohexenylthio, heptenylthio, cycloheptenylthio, octenylthio, cyclooctenylthio, ethynylthio, propynylthio, butynylthio, pentynylthio, hexynylthio, heptynylthio or octynylthio. In general, alkyl, alkoxy or thioalkyl groups according to the present invention may be straight-chain, branched or cyclic, where one or more nonadjacent CH₂ groups may be replaced by the abovementioned groups; in addition, it is also possible for one or more hydrogen atoms to be replaced by D, F, Cl, Br, I, CN or NO₂, preferably F, CI or CN, further preferably F or CN, especially preferably CN.

An aromatic or heteroaromatic ring system which has 5-60 aromatic ring atoms and may also be substituted in each case by the abovementioned R² radicals or a hydrocarbyl radical and which may be joined to the aromatic or heteroaromatic system via any desired positions is especially understood to mean groups derived from benzene, naphthalene, anthracene, benzanthracene, phenanthrene, pyrene, chrysene, perylene, fluoranthene, naphthacene, pentacene, benzopyrene, biphenyl, biphenylene, terphenyl, triphenylene, fluorene, spirobifluorene, dihydrophenanthrene, dihydropyrene, tetrahydropyrene, cis- or trans-indenofluorene, cis- or trans-indenocarbazole, cis- or trans-indolocarbazole, truxene, isotruxene, spirotruxene, spiroisotruxene, furan, benzofuran, isobenzofuran, dibenzofuran, thiophene, benzothiophene, isobenzothiophene, dibenzothiophene, pyrrole, indole, isoindole, carbazole, pyridine, quinoline, isoquinoline, acridine, phenanthridine, benzo-5,6-quinoline, benzo-6,7-quinoline, benzo-7,8-quinoline, phenothiazine, phenoxazine, pyrazole, indazole, imidazole, benzimidazole, naphthimidazole, phenanthrimidazole, pyridimidazole, pyrazinimidazole, quinoxalinimidazole, oxazole, benzoxazole, naphthoxazole, anthroxazole, phenanthroxazole, isoxazole, 1,2-thiazole, 1,3-thiazole, benzothiazole, pyridazine, hexaazatriphenylene, benzopyridazine, pyrimidine, benzopyrimidine, quinoxaline, 1,5-diazaanthracene, 2,7-diazapyrene, 2,3-diazapyrene, 1,6-diazapyrene, 1,8-diazapyrene, 4,5-diazapyrene, 4,5,9,10-tetraazaperylene, pyrazine, phenazine, phenoxazine, phenothiazine, fluorubine, naphthyridine, azacarbazole, benzocarboline, phenanthroline, 1,2,3-triazole, 1,2,4-triazole, benzotriazole, 1,2,3-oxadiazole, 1,2,4-oxadiazole, 1,2,5-oxadiazole, 1,3,4-oxadiazole, 1,2,3-thiadiazole, 1,2,4-thiadiazole, 1,2,5-thiadiazole, 1,3,4-thiadiazole, 1,3,5-triazine, 1,2,4-triazine, 1,2,3-triazine, tetrazole, 1,2,4,5-tetrazine, 1,2,3,4-tetrazine, 1,2,3,5-tetrazine, purine, pteridine, indolizine and benzothiadiazole, or groups derived from combinations of these systems.

When two R or R¹ radicals together form a ring system, it may be mono- or polycyclic, and aliphatic, heteroaliphatic, or else, for R¹ radicals, aromatic or heteroaromatic. In this case, the radicals which together form a ring system are preferably adjacent, meaning that these radicals are bonded to the same carbon atom or to carbon atoms directly bonded to one another.

The wording that two or more radicals together may form a ring, in the context of the present description, shall be understood to mean, inter alia, that the two radicals are joined to one another by a chemical bond with formal elimination of two hydrogen atoms. This is illustrated by the following scheme:

In addition, however, the abovementioned wording shall also be understood to mean that, if one of the two radicals is hydrogen, the second radical binds to the position to which the hydrogen atom was bonded, forming a ring. This shall be illustrated by the following scheme:

In a preferred embodiment of the invention, Y is NR′, O or S, more preferably NR′ or O and most preferably NR′.

Preference is thus given to a compound of one of the following formulae (1′) and (1″):

where X has the definitions given above and the compound of the formula (1″) has at least one R radical selected from the group consisting of NAr₂ and an aromatic or heteroaromatic ring system. Particular preference is given here to the compound of the formula (1′).

In a preferred embodiment of the compounds of the formula (1), (1′) or (1″), not more than two symbols X in total, preferably not more than one symbol X in total, per ring are N, and the remaining symbols X are CR. More preferably, a total of not more than two symbols X are N; in particular, not more than one symbol X is N. Most preferably, all symbols X are CR.

Preference is given to the compounds of the formulae (2a) to (2h)

where the symbols used have the definitions given above. Preference is given here to the compounds (2a) to (2f) and (2h) when Y═O, and preference is given to the compounds of the formulae (2a), (2b) and (2d) to (2h) when Y═NR′. Particular preference is given to the compounds of the formula (2a).

Particular preference is given to the compounds of the formulae (2a′) and (2a″)

where R and R′ have the definitions given above and at least one R group in formula (2a″) is selected from the group consisting of NAr₂ and an aromatic or heteroaromatic ring system.

A preferred embodiment of the compounds of the formula (2a) is the compounds of the following formula (3):

where the symbols used have the definitions given above.

Particular preference is given to the compounds of the formulae (3′) and (3″)

where R and R′ have the definitions given above and at least one R group in formula (3″) is selected from the group consisting of NAr₂ and an aromatic or heteroaromatic ring system.

A preferred embodiment of the compounds of the formula (3) is the compounds of the following formula (4);

where the symbols used have the definitions given above.

Particular preference is given to the compounds of the formulae (4′) and (4″)

where R and R′ have the definitions given above and at least one R group in formula (4″) is selected from the group consisting of NAr₂ and an aromatic or heteroaromatic ring system.

There follows a description of preferred substituents R and R′ in the compounds of the invention.

In a preferred embodiment of the invention, R is the same or different at each instance and is selected from the group consisting of H, D, F, NAr₂, CN, OR¹, a straight-chain alkyl group having 1 to 10 carbon atoms or an alkenyl group having 2 to 10 carbon atoms or a branched or cyclic alkyl group having 3 to 10 carbon atoms, where the alkyl or alkenyl group may each be substituted by one or more R¹ radicals, and where one or more nonadjacent CH₂ groups may be replaced by O, or an aromatic or heteroaromatic ring system which has 6 to 30 aromatic ring atoms and may be substituted in each case by one or more R¹ radicals; at the same time, two R radicals together may also form an aliphatic ring system. More preferably, R is the same or different at each instance and is selected from the group consisting of H, NAr₂, a straight-chain alkyl group having 1 to 6 carbon atoms, especially having 1, 2, 3 or 4 carbon atoms, or a branched or cyclic alkyl group having 3 to 6 carbon atoms, where the alkyl group in each case may be substituted by one or more R¹ radicals, but is preferably unsubstituted, or an aromatic or heteroaromatic ring system which has 6 to 24 aromatic ring atoms and may be substituted in each case by one or more preferably nonaromatic R¹ radicals. Most preferably, R is the same or different at each instance and is selected from the group consisting of H, NAr₂ or an aromatic or heteroaromatic ring system which has 6 to 24 aromatic ring atoms, especially 6 to 13 aromatic ring atoms, and may be substituted in each case by one or more preferably nonaromatic R¹ radicals.

In a further preferred embodiment of the invention, R′ is an aromatic or heteroaromatic ring system which has 6 to 30 aromatic ring atoms and may be substituted by one or more R¹ radicals. In a particularly preferred embodiment of the invention, R′ is an aromatic or heteroaromatic ring system which has 6 to 24 aromatic ring atoms, especially 6 to 13 aromatic ring atoms, and may be substituted by one or more preferably nonaromatic R¹ radicals.

In a further preferred embodiment of the invention, R¹ is the same or different at each instance and is selected from the group consisting of H, D, F, CN, OR², a straight-chain alkyl group having 1 to 10 carbon atoms or an alkenyl group having 2 to 10 carbon atoms or a branched or cyclic alkyl group having 3 to 10 carbon atoms, where the alkyl or alkenyl group may each be substituted by one or more R² radicals, and where one or more nonadjacent CH₂ groups may be replaced by O, or an aromatic or heteroaromatic ring system which has 6 to 30 aromatic ring atoms and may be substituted in each case by one or more R² radicals; at the same time, two or more R¹ radicals together may form an aliphatic ring system. In a particularly preferred embodiment of the invention, R¹ is the same or different at each instance and is selected from the group consisting of H, a straight-chain alkyl group having 1 to 6 carbon atoms, especially having 1, 2, 3 or 4 carbon atoms, or a branched or cyclic alkyl group having 3 to 6 carbon atoms, where the alkyl group may be substituted by one or more R² radicals, but is preferably unsubstituted, or an aromatic or heteroaromatic ring system which has 6 to 24 aromatic ring atoms, especially 6 to 13 aromatic ring atoms, and may be substituted in each case by one or more R² radicals, but is preferably unsubstituted.

In a further preferred embodiment of the invention, R² is the same or different at each instance and is H, an alkyl group having 1 to 4 carbon atoms or an aryl group having 6 to 10 carbon atoms, which may be substituted by an alkyl group having 1 to 4 carbon atoms, but is preferably unsubstituted.

As described above, the compound of the invention contains at least one R′ group and/or contains at least one substituent R selected from the group consisting of NAr₂ and an aromatic or heteroaromatic ring system which has 5 to 60 aromatic ring atoms and may be substituted by one or more R¹ radicals. In one embodiment of the invention, Y═NR′ and the base skeleton is unsubstituted, i.e. R═H. In a further embodiment of the invention, Y═NR′ and the base skeleton is substituted by at least one substituent selected from the group consisting of NAr₂ and an aromatic or heteroaromatic ring system which has 5 to 60 aromatic ring atoms and may be substituted by one or more R¹ radicals. There follows a description of preferred aromatic and heteroaromatic ring systems which may be present as substituent R and/or R′ or as Ar group within the NAr₂ substituent in the compound of the invention.

Suitable aromatic or heteroaromatic ring systems R, R′ or Ar′ are selected from phenyl, biphenyl, especially ortho-, meta- or para-biphenyl, terphenyl, especially ortho-, meta- or para-terphenyl or branched terphenyl, quaterphenyl, especially ortho-, meta- or para-quaterphenyl or branched quaterphenyl, fluorene which may be joined via the 1, 2, 3 or 4 position, spirobifluorene which may be joined via the 1, 2, 3 or 4 position, naphthalene, especially 1- or 2-bonded naphthalene, indole, benzofuran, benzothiophene, carbazole which may be joined via the 1, 2, 3 or 4 position, dibenzofuran which may be joined via the 1, 2, 3 or 4 position, dibenzothiophene which may be joined via the 1, 2, 3 or 4 position, indenocarbazole, indolocarbazole, pyridine, pyrimidine, pyrazine, pyridazine, triazine, phenanthrene, triphenylene or a combination of two or three of these groups, each of which may be substituted by one or more R¹ radicals.

The R, R′ and Ar groups here are preferably selected from the groups of the following formulae Ar-1 to Ar-75:

where R¹ has the definitions given above, the dotted bond represents the bond to Y or to a carbon atom of the base skeleton in formula (1) or in the preferred embodiments or to the nitrogen atom in the NAr₂ group and, in addition:

Ar¹ is the same or different at each instance and is a bivalent aromatic or heteroaromatic ring system which has 6 to 18 aromatic ring atoms and may be substituted in each case by one or more R¹ radicals;

A is the same or different at each instance and is C(R¹)₂, NR¹, O or S;

n is 0 or 1, where n=0 means that no A group is bonded at this position and R¹ radicals are bonded to the corresponding carbon atoms instead;

m is 0 or 1, where m=0 means that the Ar¹ group is absent and that the corresponding aromatic or heteroaromatic group is bonded directly to Y or a carbon atom of the base skeleton in formula (1) or in the preferred embodiments, or to the nitrogen atom in the NAr₂ group; with the proviso that m=1 for the structures (Ar-12), (Ar-17), (Ar-21), (Ar-25), (Ar-26), (Ar-30), (Ar-34), (Ar-38) and (Ar-39) when these groups are embodiments of R′ or Ar.

When the abovementioned groups for R, R′ or Ar have two or more A groups, possible options for these include all combinations from the definition of A. Preferred embodiments in that case are those in which one A group is NR¹ and the other A group is C(R¹)₂ or in which both A groups are NR¹ or in which both A groups are O. In a particularly preferred embodiment of the invention, in R, R′ or Ar groups having two or more A groups, at least one A group is C(R¹)₂ or is NR¹.

When A is NR¹, the substituent R¹ bonded to the nitrogen atom is preferably an aromatic or heteroaromatic ring system which has 5 to 24 aromatic ring atoms and may also be substituted by one or more R² radicals. In a particularly preferred embodiment, this substituent R¹ is the same or different at each instance and is an aromatic or heteroaromatic ring system which has 6 to 24 aromatic ring atoms, which does not have any fused aryl groups and which does not have any fused heteroaryl groups in which two or more aromatic or heteroaromatic 6-membered ring groups are fused directly to one another, and which may also be substituted in each case by one or more R² radicals. Particular preference is given to phenyl, biphenyl, terphenyl and quaterphenyl having bonding patterns as listed above for Ar-1 to Ar-11, where these structures may be substituted by one or more R² radicals, but are preferably unsubstituted.

When A is C(R¹)₂, the substituents R¹ bonded to this carbon atom are preferably the same or different at each instance and are a linear alkyl group having 1 to 10 carbon atoms or a branched or cyclic alkyl group having 3 to 10 carbon atoms or an aromatic or heteroaromatic ring system having 5 to 24 aromatic ring atoms, which may also be substituted by one or more R² radicals. Most preferably, R¹ is a methyl group or a phenyl group. In this case, the R¹ radicals together may also form a ring system, which leads to a spiro system.

Further suitable R, R′ or Ar groups are groups of the formula —Ar⁴—N(Ar²)(Ar³) where Ar², Ar³ and Ar⁴ are the same or different at each instance and are an aromatic or heteroaromatic ring system which has 5 to 24 aromatic ring atoms and may be substituted in each case by one or more R¹ radicals. The total number of aromatic ring atoms in Ar², Ar³ and Ar⁴ here is not more than 60 and preferably not more than 40.

In this case, Ar⁴ and Ar² may also be bonded to one another and/or Ar² and Ar³ to one another by a group selected from C(R¹)₂, NR¹, O and S. Preferably, Ar⁴ and Ar² are joined to one another and Ar² and Ar³ to one another in the respective ortho position to the bond to the nitrogen atom. In a further embodiment of the invention, none of the Ar², Ar³ and Ar⁴ groups are bonded to one another.

Preferably, Ar⁴ is an aromatic or heteroaromatic ring system which has 6 to 24 aromatic ring atoms, preferably 6 to 12 aromatic ring atoms, and may be substituted in each case by one or more R¹ radicals. More preferably, Ar⁴ is selected from the group consisting of ortho-, meta- or para-phenylene or ortho-, meta- or para-biphenyl, each of which may be substituted by one or more R¹ radicals, but are preferably unsubstituted. Most preferably, Ar⁴ is an unsubstituted phenylene group. This is especially true when Ar⁴ is bonded to Ar² via a single bond.

Preferably, Ar² and Ar³ are the same or different at each instance and are an aromatic or heteroaromatic ring system which has 6 to 24 aromatic ring atoms and may be substituted in each case by one or more R¹ radicals. Particularly preferred Ar² and Ar³ groups are the same or different at each instance and are selected from the group consisting of benzene, ortho-, meta- or para-biphenyl, ortho-, mete- or para-terphenyl or branched terphenyl, ortho-, meta- or para-quaterphenyl or branched quaterphenyl, 1-, 2-, 3- or 4-fluorenyl, 1-, 2-, 3- or 4-spirobifluorenyl, 1- or 2-naphthyl, indole, benzofuran, benzothiophene, 1-, 2-, 3- or 4-carbazole, 1-, 2-, 3- or 4-dibenzofuran, 1-, 2-, 3- or 4-dibenzothiophene, indenocarbazole, indolocarbazole, 2-, 3- or 4-pyridine, 2-, 4- or 5-pyrimidine, pyrazine, pyridazine, triazine, phenanthrene, triphenylene or combinations of two, three or four of these groups, each of which may be substituted by one or more R¹ radicals. More preferably, Ar² and Ar³ are the same or different at each instance and are an aromatic ring system which has 6 to 24 aromatic ring atoms and may be substituted by one or more R¹ radicals, especially selected from the groups consisting of benzene, biphenyl, especially ortho-, meta- or para-biphenyl, terphenyl, especially ortho-, meta- or para-terphenyl or branched terphenyl, quaterphenyl, especially ortho-, meta- or para-quaterphenyl or branched quaterphenyl, fluorene, especially 1-, 2-, 3- or 4-fluorene, or spirobifluorene, especially 1-, 2-, 3- or 4-spirobifluorene.

At the same time, in compounds of the invention that are processed by vacuum evaporation, the alkyl groups preferably have not more than five carbon atoms, more preferably not more than 4 carbon atoms, most preferably not more than 1 carbon atom. For compounds which are processed from solution, suitable compounds are also those substituted by alkyl groups, especially branched alkyl groups, having up to 10 carbon atoms or those substituted by oligoarylene groups, for example ortho-, meta- or para-terphenyl or quaterphenyl or branched terphenyl or quaterphenyl groups.

When the compounds of the formula (1) or the preferred embodiments are used as matrix material for a phosphorescent emitter or in a layer directly adjoining a phosphorescent layer, it is further preferable when the compound does not contain any fused aryl or heteroaryl groups in which more than two six-membered rings are fused directly to one another. It is especially preferable when the R, R′, Ar, R¹ and R² radicals do not contain any fused aryl or heteroaryl groups in which two or more six-membered rings are fused directly to one another. An exception to this is formed by phenanthrene and triphenylene which, because of their high triplet energy, may be preferable in spite of the presence of fused aromatic six-membered rings.

The abovementioned preferred embodiments may be combined with one another as desired within the restrictions defined in claim 1. In a particularly preferred embodiment of the invention, the abovementioned preferences occur simultaneously.

Examples of preferred compounds according to the embodiments detailed above are the compounds detailed in the following table:

The synthesis of the unsubstituted base skeleton is known from the literature and can be effected, for example, according to D. J. Hagan et al. (J. Chem. Soc., Perkin Transactions 1: Organic and Bio-Organic Chemistry, 1997, (18), 2739-2746) or according to J. Stanslas et al. (J. Med. Chem., 2000, 43(8), 1563-1572).

The compounds of the invention can be prepared from the 8H-quino[4,3,2-kl]acridines and halogenated electron-deficient heterocycles (triazines, pyrimidines, pyridines, pyrazoles, imidazoles, etc.) using a strong base (NaH, Na/K alkoxides, alkyl-/aryllithium compounds, etc.), as shown in scheme 1 below.

The compounds of the invention can also be prepared from the 8H-quino[4,3,2-kl]acridines and halogenated aromatics/heteroaromatics by palladium- or copper-catalyzed Buchwald coupling or Ullmann-analogous coupling using a base, as shown in scheme 2 below.

Halogen- or triflate-functionalized 8H-quino[4,3,2-kl]acridines, [1]benzopyrano[4,3,2-gh]phenanthridines or [1]benzothiopyrano[4,3,2-gh]phenanthridines can be further functionalized by methods familiar to the person skilled in the art, for example via Suzuki coupling, as shown in scheme 3 below.

Halogen- or triflate-functionalized 8H-quino[4,3,2-kl]acridines, [1]benzopyrano[4,3,2-gh]phenanthridines or [1]benzothiopyrano[4,3,2-gh]phenanthridines can be further functionalized with secondary amines or carbazoles by methods familiar to the person skilled in the art, for example via Buchwald or Ullmann coupling, as shown in scheme 4.

It is of course possible in an entirely analogous manner to use di-, tri- or oligohalogen-functionalized reactants in the abovementioned reactions.

The present invention further provides a process for preparing a compound of formula (1) or the preferred embodiments, comprising the reaction steps of:
a) providing the base skeleton having a reactive leaving group; and
b) coupling an aromatic or heteroaromatic ring system or a compound H—NAr₂ to the base skeleton with detachment of the leaving group.

Suitable leading groups are, for example, halogen, triflate or mesylate, but also hydrogen when the coupling in step B is effected on the Y group (when Y═NR′).

For the processing of the compounds of the invention from a liquid phase, for example by spin-coating or by printing methods, formulations of the compounds of the invention are required. These formulations may, for example, be solutions, dispersions or emulsions. For this purpose, it may be preferable to use mixtures of two or more solvents. Suitable and preferred solvents are, for example, toluene, anisole, o-, m- or p-xylene, methyl benzoate, mesitylene, tetralin, veratrole, THF, methyl-THF, THP, chlorobenzene, dioxane, phenoxytoluene, especially 3-phenoxytoluene, (−)-fenchone, 1,2,3,5-tetramethylbenzene, 1,2,4,5-tetramethylbenzene, 1-methylnaphthalene, 2-methylbenzothiazole, 2-phenoxyethanol, 2-pyrrolidinone, 3-methylanisole, 4-methylanisole, 3,4-dimethylanisole, 3,5-dimethylanisole, acetophenone, α-terpineol, benzothiazole, butyl benzoate, cumene, cyclohexanol, cyclohexanone, cyclohexylbenzene, decalin, dodecylbenzene, ethyl benzoate, indane, NMP, p-cymene, phenetole, 1,4-diisopropylbenzene, dibenzyl ether, diethylene glycol butyl methyl ether, triethylene glycol butyl methyl ether, diethylene glycol dibutyl ether, triethylene glycol dimethyl ether, diethylene glycol monobutyl ether, tripropylene glycol dimethyl ether, tetraethylene glycol dimethyl ether, 2-isopropylnaphthalene, pentylbenzene, hexylbenzene, heptylbenzene, octylbenzene, 1,1-bis(3,4-dimethylphenyl)ethane, 2-methyl biphenyl, 3-methylbiphenyl, 1-methylnaphthalene, 1-ethylnaphthalene, ethyl octanoate, diethyl sebacate, octyl octanoate, heptylbenzene, menthyl isovalerate, cyclohexyl hexanoate or mixtures of these solvents.

The present invention therefore further provides a formulation comprising a compound of the invention and at least one further compound. The further compound may, for example, be a solvent, especially one of the abovementioned solvents or a mixture of these solvents. The further compound may alternatively be at least one further organic or inorganic compound which is likewise used in the electronic device, for example an emitting compound and/or a further matrix material. Suitable emitting compounds and further matrix materials are listed at the back in connection with the organic electroluminescent device. This further compound may also be polymeric.

The compounds of the invention are suitable for use in an electronic device, especially in an organic electroluminescent device.

The present invention therefore further provides for the use of a compound of the invention in an electronic device, especially in an organic electroluminescent device.

The present invention still further provides an electronic device comprising at least one compound of the invention.

An electronic device in the context of the present invention is a device comprising at least one layer comprising at least one organic compound. This component may also comprise inorganic materials or else layers formed entirely from inorganic materials.

The electronic device is preferably selected from the group consisting of organic electroluminescent devices (OLEDs), organic integrated circuits (O-ICs), organic field-effect transistors (O-FETs), organic thin-film transistors (O-TFTs), organic light-emitting transistors (O-LETs), organic solar cells (O-SCs), dye-sensitized organic solar cells (DSSCs), organic optical detectors, organic photoreceptors, organic field-quench devices (O-FQDs), light-emitting electrochemical cells (LECs), organic laser diodes (O-lasers) and organic plasmon emitting devices, but preferably organic electroluminescent devices (OLEDs), more preferably phosphorescent OLEDs.

The organic electroluminescent device comprises cathode, anode and at least one emitting layer. Apart from these layers, it may also comprise further layers, for example in each case one or more hole injection layers, hole transport layers, hole blocker layers, electron transport layers, electron injection layers, exciton blocker layers, electron blocker layers and/or charge generation layers. It is likewise possible for interlayers having an exciton-blocking function, for example, to be introduced between two emitting layers. However, it should be pointed out that not necessarily every one of these layers need be present. In this case, it is possible for the organic electroluminescent device to contain an emitting layer, or for it to contain a plurality of emitting layers. If a plurality of emission layers are present, these preferably have several emission maxima between 380 nm and 750 nm overall, such that the overall result is white emission; in other words, various emitting compounds which may fluoresce or phosphoresce are used in the emitting layers. Especially preferred are systems having three emitting layers, where the three layers show blue, green and orange or red emission. The organic electroluminescent device of the invention may also be a tandem OLED, especially for white-emitting OLEDs.

The compound of the invention according to the above-detailed embodiments may be used in different layers, according to the exact structure. Preference is given to an organic electroluminescent device comprising a compound of formula (1) or the above-recited preferred embodiments in an emitting layer as matrix material for phosphorescent emitters or for emitters that exhibit TADF (thermally activated delayed fluorescence), especially for phosphorescent emitters. In this case, the organic electroluminescent device may contain an emitting layer, or it may contain a plurality of emitting layers, where at least one emitting layer contains at least one compound of the invention as matrix material. In addition, the compound of the invention can also be used in an electron transport layer and/or in a hole transport layer and/or in an exciton blocker layer and/or in a hole blocker layer. Especially when Y═O, preference is also given to use in an electron transport layer or hole blocker layer.

When the compound of the invention is used as matrix material for a phosphorescent compound in an emitting layer, it is preferably used in combination with one or more phosphorescent materials (triplet emitters). Phosphorescence in the context of this invention is understood to mean luminescence from an excited state having higher spin multiplicity, i.e. a spin state >1, especially from an excited triplet state. In the context of this application, all luminescent complexes with transition metals or lanthanides, especially all iridium, platinum and copper complexes, shall be regarded as phosphorescent compounds.

The mixture of the compound of the invention and the emitting compound contains between 99% and 1% by volume, preferably between 98% and 10% by volume, more preferably between 97% and 60% by volume and especially between 95% and 80% by volume of the compound of the invention, based on the overall mixture of emitter and matrix material. Correspondingly, the mixture contains between 1% and 99% by volume, preferably between 2% and 90% by volume, more preferably between 3% and 40% by volume and especially between 5% and 20% by volume of the emitter, based on the overall mixture of emitter and matrix material.

A further preferred embodiment of the present invention is the use of the compound of the invention as matrix material for a phosphorescent emitter in combination with a further matrix material. Suitable matrix materials which can be used in combination with the inventive compounds are aromatic ketones, aromatic phosphine oxides or aromatic sulfoxides or sulfones, for example according to WO 2004/013080, WO 2004/093207, WO 2006/005627 or WO 2010/006680, triarylamines, carbazole derivatives, e.g. CBP (N,N-biscarbazolylbiphenyl) or the carbazole derivatives disclosed in WO 2005/039246, US 2005/0069729, JP 2004/288381, EP 1205527, WO 2008/086851 or WO 2013/041176, indolocarbazole derivatives, for example according to WO 2007/063754 or WO 2008/056746, indenocarbazole derivatives, for example according to WO 2010/136109, WO 2011/000455, WO 2013/041176 or WO 2013/056776, azacarbazole derivatives, for example according to EP 1617710, EP 1617711, EP 1731584, JP 2005/347160, bipolar matrix materials, for example according to WO 2007/137725, silanes, for example according to WO 2005/111172, azaboroles or boronic esters, for example according to WO 2006/117052, triazine derivatives, for example according to WO 2007/063754, WO 2008/056746, WO 2010/015306, WO 2011/057706, WO 2011/060859 or WO 2011/060877, zinc complexes, for example according to EP 652273 or WO 2009/062578, diazasilole or tetraazasilole derivatives, for example according to WO 2010/054729, diazaphosphole derivatives, for example according to WO 2010/054730, bridged carbazole derivatives, for example according to WO 2011/042107, WO 2011/060867, WO 2011/088877 and WO 2012/143080, triphenylene derivatives, for example according to WO 2012/048781, or dibenzofuran derivatives, for example according to WO 2015/169412, WO 2016/015810, WO 2016/023608 or the as yet unpublished applications EP 16158460.2 or EP 16159829.7. It is likewise possible for a further phosphorescent emitter having shorter-wavelength emission than the actual emitter to be present as co-host in the mixture, or a compound not involved in charge transport to a significant extent, if at all, as described, for example, in WO 2010/108579.

Especially suitable in combination with the compound of the invention as co-matrix material are compounds which have a large bandgap and themselves take part at least not to a significant degree, if any at all, in the charge transport of the emitting layer. Such materials are preferably pure hydrocarbons. Examples of such materials can be found, for example, in WO 2009/124627 or in WO 2010/006680.

Suitable phosphorescent compounds (=triplet emitters) are especially compounds which, when suitably excited, emit light, preferably in the visible region, and also contain at least one atom of atomic number greater than 20, preferably greater than 38 and less than 84, more preferably greater than 56 and less than 80, especially a metal having this atomic number. Preferred phosphorescence emitters used are compounds containing copper, molybdenum, tungsten, rhenium, ruthenium, osmium, rhodium, iridium, palladium, platinum, silver, gold or europium, especially compounds containing iridium or platinum.

Examples of the above-described emitters can be found in applications WO 00/70655, WO 2001/41512, WO 2002/02714, WO 2002/15645, EP 1191613, EP 1191612, EP 1191614, WO 05/033244, WO 05/019373, US 2005/0258742, WO 2009/146770, WO 2010/015307, WO 2010/031485, WO 2010/054731, WO 2010/054728, WO 2010/086089, WO 2010/099852, WO 2010/102709, WO 2011/032626, WO 2011/066898, WO 2011/157339, WO 2012/007086, WO 20141008982, WO 2014/023377, WO 2014/094961, WO 2014/094960, WO 2015/036074, WO 2015/104045, WO 2015/117718, WO 2016/015815, WO 2016/124304, WO 2017/032439 and the as yet unpublished application EP16179378.1. In general, all phosphorescent complexes as used for phosphorescent OLEDs according to the prior art and as known to those skilled in the art in the field of organic electroluminescence are suitable, and the person skilled in the art will be able to use further phosphorescent complexes without exercising inventive skill.

Explicit examples of phosphorescent dopants are adduced in the following table:

The compounds of the invention are especially also suitable as matrix materials for phosphorescent emitters in organic electroluminescent devices, as described, for example, in WO 98/24271, US 2011/0248247 and US 2012/0223633. In these multicolor display components, an additional blue emission layer is applied by vapor deposition over the full area to all pixels, including those having a color other than blue. It has been found that, surprisingly, the compounds of the invention, when they are used as matrix materials for the red and/or green pixels, still lead to very good emission together with the blue emission layer applied by vapor deposition.

In a further embodiment of the invention, the organic electroluminescent device of the invention does not contain any separate hole injection layer and/or hole transport layer and/or hole blacker layer and/or electron transport layer, meaning that the emitting layer directly adjoins the hole injection layer or the anode, and/or the emitting layer directly adjoins the electron transport layer or the electron injection layer or the cathode, as described, for example, in WO 2005/053051. It is additionally possible to use a metal complex identical or similar to the metal complex in the emitting layer as hole transport or hole injection material directly adjoining the emitting layer, as described, for example, in WO 2009/030981.

In a further embodiment of the invention, the compound of the invention is used in a hole blocker layer or an electron transport layer. Especially suitable for this purpose are compounds in which Y═O and/or in which at least one symbol X is N and/or which have at least one substituent R or R′ which is an electron-deficient heteroaryl group or contains an electron-deficient heteroaryl group, for example triazine or pyrimidine.

In the further layers of the organic electroluminescent device of the invention, it is possible to use any materials as typically used according to the prior art. The person skilled in the art will therefore be able, without exercising inventive skill, to use any materials known for organic electroluminescent devices in combination with the inventive compounds of formula (1) or the above-recited preferred embodiments.

Additionally preferred is an organic electroluminescent device, characterized in that one or more layers are coated by a sublimation process. In this case, the materials are applied by vapor deposition in vacuum sublimation systems at an initial pressure of less than 10⁻⁵ mbar, preferably less than 10⁻⁶ mbar. However, it is also possible that the initial pressure is even lower, for example less than 10⁻⁷ mbar.

Preference is likewise given to an organic electroluminescent device, characterized in that one or more layers are coated by the OVPD (organic vapor phase deposition) method or with the aid of a carrier gas sublimation. In this case, the materials are applied at a pressure between 10⁻⁵ mbar and 1 bar. A special case of this method is the OVJP (organic vapor jet printing) method, in which the materials are applied directly by a nozzle and thus structured.

Preference is additionally given to an organic electroluminescent device, characterized in that one or more layers are produced from solution, for example by spin-coating, or by any printing method, for example screen printing, flexographic printing, offset printing, LITI (light-induced thermal imaging, thermal transfer printing), inkjet printing or nozzle printing. For this purpose, soluble compounds are needed, which are obtained, for example, through suitable substitution.

In addition, hybrid methods are possible, in which, for example, one or more layers are applied from solution and one or more further layers are applied by vapor deposition.

These methods are known in general terms to those skilled in the art and can be applied by those skilled in the art without exercising inventive skill to organic electroluminescent devices comprising the compounds of the invention.

The compounds of the invention and the organic electroluminescent devices of the invention are notable for one or more of the following surprising advantages over the prior art:
1. The compounds of the invention, used as matrix material for phosphorescent emitters, lead to long lifetimes.
2. The compounds of the invention lead to high efficiencies. This is especially true when the compounds are used as matrix material for a phosphorescent emitter.
3. The compounds of the invention lead to low operating voltages. This is especially true when the compounds are used as matrix material for a phosphorescent emitter.
4. The compounds of the invention, especially compounds in which Y═O and/or in which at least one symbol X is N and/or which have at least one electron-deficient heteroaromatic substituent lead to very good results when used in a hole blocker layer or an electron transport layer.

These abovementioned advantages are not accompanied by a deterioration in the further electronic properties.

The invention is illustrated in more detail by the examples which follow, without any intention of restricting it thereby. The person skilled in the art will be able to use the information given to execute the invention over the entire scope disclosed and to prepare further compounds of the invention without exercising inventive skill and to use them in electronic devices or to employ the process of the invention.

### EXAMPLES

The syntheses which follow, unless stated otherwise, are conducted under a protective gas atmosphere in dried solvents. The metal complexes are additionally handled with exclusion of light or under yellow light. The solvents and reagents can be purchased, for example, from Sigma-ALDRICH or ABCR. The respective figures in square brackets or the numbers quoted for individual compounds relate to the CAS numbers of the compounds known from the literature.

### Synthons and Inventive Examples S

### Example S1

A mixture of 30.3 g (100 mmol) of 3-chloro-8H-quino[4,3,2-kl]acridine [198025-90-0], 13.4 g (110 mmol) of phenylboronic acid [98-80-6], 69.1 g (300 mmol) of tripotassium phosphate monohydrate, 821 mg (2 mmol) of SPhos [657408-07-6], 225 (1 mmol) of palladium acetate, 50 g of glass beads (diameter 3 mm) and 300 ml of DMSO is heated to 120° C. for 24 h. After cooling, the DMSO is largely removed under reduced pressure, the residue is taken up in 500 ml of dichloromethane (DCM), and the organic phase is washed three times with 300 ml each time of water and once with 300 ml of saturated sodium chloride solution and dried over magnesium sulfate. The desiccant is filtered off with suction using a short Celite bed and the filtrate is concentrated to dryness. The crude product is recrystallized from dimethylacetamide. Yield: 24.8 g (72 mmol), 72%; purity: >98% by HPLC.

In an analogous manner, it is possible to prepare the following compounds:

| Ex. | Reactants | Product | Yield |
|---|---|---|---|
| S2 | 198025-90-0 5122-95-2 | | 68% |
| S3 | 198025-90-0 796071-96-0 | | 70% |
| S4 | 1416857-89-0 1313018-07-3 | Recrystallization from DMAC Fractional sublimation p about 10⁻⁵ mbar/T about 320° C. | 49% |
| S5 | 1416857-89-0 1510788-86-9 | Recrystallization from DMAC Fractional sublimation p about 10⁻⁵ mbar/T about 330° C. | 52% |
| S6 | 1416857-62-9 1899872-63-9 | Recrystallization from DMF Fractional sublimation p about 10⁻⁵ mbar/T about 330° C. | 55% |
| S7 | 1416942-12-5 | Recrystallization from DMF Fractional sublimation p about 10⁻⁵ mbar/T about 330° C. | 47% |
| | 1357572-68-9 | | |

### Example 1

To a solution of 26.8 g (100 mmol) of 8H-quino[4,3,2-kl]acridine [111180-95-5] and 26.8 g (100 mmol) of chlorodiphenyltriazine [3842-55-5] in 300 ml of dimethylacetamide (DMAC) are added 2.4 g (100 mmol) of sodium hydride in portions at room temperature (caution: evolution of hydrogen!). After the addition has ended, the mixture is heated gradually to 140° C. and stirring is continued until the reactants have been consumed (about 2 h). After cooling, the yellow acicular solids are filtered off with suction and washed once with 100 ml of DMAC and three times with 100 ml each time of ethanol, and then dried under reduced pressure. The crude product thus obtained is recrystallized five times from DMAC and then fractionally sublimed twice (p about 10⁻⁵ mbar, T=about 320° C.). Yield: 26.0 g (52 mmol), 52%; purity: >99.99% by HPLC.

In an analogous manner, it is possible to prepare the following compounds:

| Ex. | Reactants | Product | Yield |
|---|---|---|---|
| 2 | 111180-95-5 2915-16-4 | | 56% |
| 3 | 111180-95-5 1453806-51-3 | | 49% |
| 4 | 111180-95-5 1624289-88-8 | | 55% |
| 5 | 111180-95-5 162429349-7 | | 61% |
| 6 | 111180-95-5 1384480-16-3 | | 57% |
| 7 | 111180-95-5 1621467-31-9 | | 53% |
| 8 | 111180-95-5 1621467-62-2 | | 60% |
| 9 | 111180-95-5 4786-80-5 | | 64% |
| 10 | 111180-95-5 1821393-98-9 | | 58% |
| 11 | 111180-95-5 1821394-04-0 | | 55% |
| 12 | 111180-95-5 1421599-32-7 | | 61% |
| 13 | 198025-92-2 | | 52% |
| | 1421599-32-7 | | |
| 14 | 198025-93-3 | | 56% |
| | 1421599-30-5 | | |
| 15 | 111180-95-5 1417522-89-4 | | 56% |
| 16 | 111180-95-5 1333505-17-1 | | 59% |
| 17 | 111180-95-5 1592541-57-5 | | 60% |
| 18 | 111180-95-5 1835205-67-8 | | 54% |
| 19 | 111180-95-5 1300115-09-6 | | 62% |
| 20 | 111180-95-5 1689476-03-1 | | 59% |
| 21 | 111180-95-5 1616231-57-2 | | 55% |
| 22 | 111180-95-5 1616231-59-4 | | 49% |
| 23 | 111180-95-5 1836145-06-2 | | 60% |
| 24 | S1 1883265-32-4 | | 57% |
| 25 | S2 1472729-25-1 | | 56% |
| 26 | S2 1616231-53-8 | | 47% |
| 27 | 111180-95-5 1616231-53-8 | | 51% |

### Example 100

A mixture of 26.8 g (100 mmol) of 8H-quino[4,3,2-kl]acridine [111180-95-5], 35.6 g (100 mmol) of 5′-iodo-1,1′:3′,1″-terphenyl [87666-86-2], 27.6 g (200 mmol) of potassium carbonate, 28.4 g (200 mmol) of sodium sulfate, 1.3 g (20 mmol) of copper powder, 100 g of glass beads (diameter 3 mm) and 300 ml of nitrobenzene is heated under reflux with good stirring for 16 h. After cooling, 1000 ml of methanol are added, and the solids are filtered off with suction and washed three times with 300 ml each time of methanol. The solids are subjected to hot extraction by stirring in 1000 ml of water, filtered off with suction and then washed twice with 200 ml each time of hot water and three times with 200 ml each time of methanol, and then dried under reduced pressure. The crude product thus obtained is hot extracted five times with o-xylene (amount initially charged 250 ml) and then fractionally sublimed twice (p about 10⁻⁵ mbar, T about 310° C.). Yield: 28.3 g (57 mmol), 57%; purity: >99.99% by HPLC.

In an analogous manner, it is possible to prepare the following compounds:

| Ex. | Reactants | Product | Yield |
|---|---|---|---|
| 101 | 87666-86-2 1777801-89-4 | | 46% |
| 102 | 87666-86-2 955959-84-9 | | 49% |
| 103 | S2 1161009-88-6 | | 45% |
| 104 | S3 502161-03-7 | | 56% |
| 105 | 87666-86-2 1369587-63-2 | | 50% |
| 106 | 87666-86-2 31037-00-0 | | 39% |
| 107 | 1416857-89-0 1257220-47-5 | | 52% |
| 108 | 1416857-89-0 1416942-12-5 | | 48% |

Production of the OLEDs

Examples I1 to I22 which follow present the use of various materials of the invention in OLEDs.

### Pretreatment for Examples I1-I22

Glass plaques coated with structured ITO (indium tin oxide) of thickness 50 nm are treated prior to coating with an oxygen plasma, followed by an argon plasma. These plasma-treated glass plaques form the substrates to which the OLEDs are applied.

The OLEDs basically have the following layer structure: substrate/hole injection layer (HIL)/hole transport layer (HTL)/electron blocker layer (EBL)/emission layer (EML)/optional hole blocker layer (HBL)/electron transport layer (ETL)/optional electron injection layer (EIL) and finally a cathode. The cathode is formed by an aluminum layer of thickness 100 nm. The exact structure of the OLEDs can be found in Table 1. The materials required for production of the OLEDs are shown in Table 2.

All materials are applied by thermal vapor deposition in a vacuum chamber. In this case, the emission layer always consists of at least one matrix material (host material) and an emitting dopant (emitter) which is added to the matrix material(s) in a particular proportion by volume by co-evaporation. Details given in such a form as IC5:IC3:TEG2 (55%:35%:10%) mean here that the material IC5 is present in the layer in a proportion by volume of 55%, IC3 in a proportion of 35% and TEG2 in a proportion of 10%. Analogously, the electron transport layer may also consist of a mixture of two materials.

The OLEDs are characterized in a standard manner. For this purpose, the electroluminescence spectra, the current efficiency (measured in cd/A), the power efficiency (measured in lm/W) and the external quantum efficiency (EQE, measured in percent) as a function of luminance, calculated from current-voltage-luminance characteristics (IUL characteristics) assuming Lambertian emission characteristics, and also the lifetime are determined. The electroluminescence spectra are determined at a luminance of 1000 cd/m², and the CIE 1931 x and y color coordinates are calculated therefrom.

Use of Mixtures of the Invention in the Emission Layer of Phosphorescent OLEDs

The materials of the invention can be used in the emission layer in phosphorescent red OLEDs. The inventive material EG1 is used in Example I1 as matrix material in combination with the phosphorescent emitter TEG5. At a luminance of 1000 cd/m², the OLED from I1 has a voltage of 3.8 V, an EQE of 21% and color coordinates of CIEx=0.67 and CIEy=0.33.

In examples I2 to I16 too, the OLED emits light with the color coordinates CIEx=0.67 and CIEy=0.33. This shows that the inventive compounds IV1-IV16 are suitable for use as matrix material in OLEDs.

Use of Mixtures of the Invention in the Electron Transport Layer of Phosphorescent OLEDs

The materials of the invention can also be used in the electron transport layer in OLEDs. In Examples I17 to I22, the inventive materials IV17 to IV22 are used in the electron transport layer. In examples I17 to I22, the OLED emits light with the color coordinates CIEx=0.67 and CIEy=0.33. This shows that the inventive compounds IV17 to IV22 are suitable for use as electron transport material in OLEDs.

TABLE 1
Structure of the OLEDs

| | HIL | HTL | EBL | EML | HBL | ETL | EIL |
|---|---|---|---|---|---|---|---|
| Ex. | thickness | thickness | thickness | thickness | thickness | thickness | thickness |
| I1 | HATCN | SpMA1 | SpMA3 | IV1:TER5 | | ST2:LiQ (50%:50%) | |
| | 5 nm | 125 nm | 10 nm | (95%:5%) 40 nm | | 35 nm | |
| I2 | HATCN | SpMA1 | SpMA3 | IV2:TER5 | | ST2:LiQ (50%:50%) | |
| | 5 nm | 125 nm | 10 nm | (95%:5%) 40 nm | | 35 nm | |
| I3 | HATCN | SpMA1 | SpMA3 | IV1:IV3:TER5 | | ST2:LiQ (50%:50%) | |
| | 5 nm | 125 nm | 10 nm | (85% 10%:5%) 40 nm | | 35 nm | |
| I4 | HATCN | SpMA1 | SpMA3 | IV1:IV4:TER5 | | ST2:LiQ (50%.50%) | |
| | 5 nm | 125 nm | 10 nm | (85% 10%:5%) 40 nm | | 35 nm | |
| I5 | HATCN | SpMA1 | SpMA3 | IV5:TER5 | | ST2:LiQ (50%.50%) | |
| | 5 nm | 125 nm | 10 nm | (95%:5%) 40 nm | | 35 nm | |
| I6 | HATCN | SpMA1 | SpMA3 | IV6:TER5 | | ST2:LiQ (50%:50%) | |
| | 5 nm | 125 nm | 10 nm | (96%:5%)40 nm | | 35 nm | |
| I7 | HATCN | SpMA1 | SpMA3 | IV7:TER5 | | ST2:LiQ (50%:50%) | |
| | 5 nm | 125 nm | 10 nm | (95%:5%) 40 nm | | 35 nm | |
| I8 | HATCN | SpMA1 | SpMA3 | IV8:TER5 | | ST2:LiQ (50%:50%) | |
| | 5 nm | 125 nm | 10 nm | (95%:5%) 40 nm | | 35nm | |
| I9 | HATCN | SpMA1 | SpMA3 | IV9:TER5 | | ST2:LiQ (50%:50%) | |
| | 5 nm | 125 nm | 10 nm | (95%:5%) 40 nm | | 35 nm | |
| I10 | HATCN | SpMA1 | SpMA3 | IV10:TER5 | | ST2:LiQ (50%:50%) | |
| | 5 nm | 125 nm | 10 nm | (95%:5%) 40 nm | | 35nm | |
| I11 | HATCN | SpMA1 | SpMA3 | IV11:TER5 | | ST2:LiQ (50%:50%) | |
| | 5 nm | 125 nm | 10 nm | (95%:5%) 40 nm | | 35nm | |
| I12 | HATCN | SpMA1 | SpMA3 | IV12:TER5 | | ST2:LiQ (50%:50%) | |
| | 5 nm | 125 nm | 10 nm | (95%:5%) 40 nm | | 35nm | |
| I13 | HATCN | SpMA1 | SpMA3 | IV1:IV13:TER5 | | ST2:LiQ (50%:50%) | |
| | 5 nm | 125 nm | 10 nm | (85% 10%:5%) | | 35nm | |
| I14 | HATCN | SpMA1 | SpMA3 | IV1:IV14:TER5 | | ST2:LiQ (50%:50%) | |
| | 5 nm | 125 nm | 10 nm | (85% 10%:5%) 40 nm | | 35nm | |
| I15 | HATCN | SpMA1 | SpMA3 | IV1:IV15:TER5 | | ST2:LiQ (50%:50%) | |
| | 5 nm | 125 nm | 10 nm | (85% 10%:5%) 40 nm | | 35nm | |
| I16 | HATCN | SpMA1 | SpMA3 | IV1:IV16:TER5 | | ST2:LiQ (50%:50%) | |
| | 5 nm | 125 nm | 10 nm | (85% 10%:5%) 40 nm | | 35nm | |
| I17 | HATCN | SpMA1 | SpMA3 | IV1:TER5 | ST2 | ST2:IV17 (50%:50%) | LiQ |
| | 5 nm | 125 nm | 10 nm | (95%:5%) 40 nm | 5 nm | 30 nm | 3 nm |
| I18 | HATCN | SpMA1 | SpMA3 | IV1.TER5 | ST2 | ST2:IV18 (50%:50%) | LiQ |
| | 5 nm | 125 nm | 10 nm | (95%:5%) 40 nm | 5 nm | 30 nm | 3 nm |
| I19 | HATCN | SpMA1 | SpMA3 | IV1:TER5 | ST2 | ST2:IV19 (50%:50%) | LiQ |
| | 5 nm | 125 nm | 10 nm | (95%:5%) 40 nm | 5 nm | 30 nm | 3 nm |
| I20 | HATCN | SpMA1 | SpMA3 | IV1:TER5 | ST2 | ST2:IV20 (50%:50%) | LiQ |
| | 5 nm | 125 nm | 10 nm | (95%:5%) 40 nm | 5 nm | 30 nm | 3 nm |
| I21 | HATCN | SpMA1 | SpMA3 | IV1:TER5 | ST2 | ST2:IV21 (50%:50%) | LiQ |
| | 5 nm | 125 nm | 10 nm | (95%:5%) 40 nm | 5 nm | 30 nm | 3 nm |
| I22 | HATCN | SpMA1 | SpMA3 | IV1:TER5 | ST2 | ST2:IV22 (50%:50%) | LiQ |
| | 5 nm | 125 nm | 10 nm | (95%:5%) 40 nm | 5 nm | 30 nm | 3 nm |

```
TABLE 2
Structural formulae of the materials for the OLEDs
  HATCN
  SpMA1
  SpMA3
  ST2
  TER5
  LiQ
  IV1
  IV2
  IV3
  IV4
  IV5
  IV6
  IV7
  IV8
  IV9
  IV10
  IV11
  IV12
  IV13
  IV14
  IV15
  IV16
  IV17
  IV18
  IV19
  IV20
  IV21
  IV22
```

1.-15. (canceled)

16. A compound of formula (1)
where the symbols used are as follows:
X is the same or different at each instance and is CR or N;
Y is NR′, BR′, O or S;
R is the same or different at each instance and is H, D, F, Cl, Br, I, NAr₂, N(R¹)₂, CN, NO₂, OR¹, SR¹, COOR¹, C(═O)N(R¹)₂, Si(R¹)₃, B(OR¹)₂, C(═O)R¹, P(═O)(R¹)₂, S(═O)R¹, S(═O)₂R¹, OSO₂R¹, a straight-chain alkyl group having 1 to 20 carbon atoms or an alkenyl or alkynyl group having 2 to 20 carbon atoms or a branched or cyclic alkyl group having 3 to 20 carbon atoms, where the alkyl, alkenyl or alkynyl group may in each case be substituted by one or more R¹ radicals and where one or more nonadjacent CH₂ groups may be replaced by Si(R¹)₂, C═O, NR¹, O, S or CONR¹, or an aromatic or heteroaromatic ring system which has 5 to 60 aromatic ring atoms and may be substituted in each case by one or more R¹ radicals; at the same time, two R radicals together may also form an aliphatic or heteroaliphatic ring system;
R′ is an aromatic or heteroaromatic ring system which has 5 to 40 aromatic ring atoms and may be substituted by one or more R¹ radicals;
Ar is the same or different at each instance and is an aromatic or heteroaromatic ring system which has 5 to 40 aromatic ring atoms and may be substituted by one or more R¹ radicals;
R¹ is the same or different at each instance and is H, D, F, Cl, Br, I, N(R²)₂, CN, NO₂, OR², SR², Si(R²)₃, B(OR²)₂, C(═O)R², P(═O)(R²)₂, S(═O)R², S(═O)₂R², OSO₂R², a straight-chain alkyl group having 1 to 20 carbon atoms or an alkenyl or alkynyl group having 2 to 20 carbon atoms or a branched or cyclic alkyl group having 3 to 20 carbon atoms, where the alkyl, alkenyl or alkynyl group may in each case be substituted by one or more R² radicals and where one or more nonadjacent CH₂ groups may be replaced by Si(R²)₂, C═O, NR², O, S or CONR², or an aromatic or heteroaromatic ring system which has 5 to 40 aromatic ring atoms and may be substituted in each case by one or more R² radicals; at the same time, two or more R¹ radicals together may form a ring system;
R² is the same or different at each instance and is H, D, F or an aliphatic, aromatic or heteroaromatic organic radical having 1 to 20 carbon atoms, in which one or more hydrogen atoms may also be replaced by F;
with the proviso that the compound of the formula (1) contains at least one substituent R′ and/or that the compound of the formula (1) contains at least one substituent R selected from the group consisting of NAr₂ and an aromatic or heteroaromatic ring system which has 5 to 60 aromatic ring atoms and may be substituted by one or more R¹ radicals.

17. The compound as claimed in claim 16, wherein the compound of formula (1) is a compound of formula (1′) or (1″)
where the symbols have the definitions given in claim 16 and the compound of the formula (1″) has at least one R radical selected from the group consisting of NAr₂ and an aromatic or heteroaromatic ring system which has 5 to 60 aromatic ring atoms and may be substituted by one or more R¹ radicals.

18. The compound as claimed in claim 16, wherein not more than one symbol X per ring is N and the remaining symbols X are CR.

19. The compound as claimed in claim 16, wherein the compound of formula (1) is a compound of one of the formulae (2a) to (2h)
where the symbols have the definitions given in claim 16.

20. The compound as claimed in claim 16 wherein the compound of formula (1) is a compound of one of the formulae (2a′) and (2a″)
where the symbols have the definitions given in claim 16 and at least one R group in formula (2a″) is selected from the group consisting of NAr₂ and an aromatic or heteroaromatic ring system which has 5 to 60 aromatic ring atoms and may be substituted by one or more R¹ radicals.

21. The compound as claimed in claim 16, wherein the compound of formula (1) is a compound of the formula (3)
where the symbols have the definitions given in claim 16.

22. The compound as claimed in claim 16, wherein the compound of formula (1) is a compound of one of the formulae (3′) and (3″)
where the symbols have the definitions given in claim 16 and at least one R group in formula (3″) is selected from the group consisting of NAr₂ and an aromatic or heteroaromatic ring system which has 5 to 60 aromatic ring atoms and may be substituted by one or more R¹ radicals.

23. The compound as claimed in claim 16, wherein the compound of formula (1) is a compound of formula (4)
where the symbols have the definitions given in claim 16.

24. The compound as claimed in claim 16, wherein the compound of formula (1) is a compound of one of the formulae (4′) and (4″)
where the symbols have the definitions given in claim 16 and at least one R group in formula (4″) is selected from the group consisting of NAr₂ and an aromatic or heteroaromatic ring system which has 5 to 60 aromatic ring atoms and may be substituted by one or more R¹ radicals.

25. The compound as claimed in claim 16, wherein R, when it is an aromatic or heteroaromatic ring system, R′ and/or Ar are selected from the group consisting of phenyl, biphenyl, terphenyl, quaterphenyl, fluorene, spirobifluorene, naphthalene, indole, benzofuran, benzothiophene, carbazole, dibenzofuran, dibenzothiophene, indenocarbazole, indolocarbazole, pyridine, pyrimidine, pyrazine, pyridazine, triazine, phenanthrene, triphenylene or a combination of two or three of these groups, each of which may be substituted by one or more R¹ radicals.

26. A process for preparing the compound as claimed in claim 16, comprising the reaction steps of:
a) providing the base skeleton having a reactive leaving group; and
b) coupling an aromatic or heteroaromatic ring system or a compound H—NAr₂ to the base skeleton with detachment of the leaving group.

27. A formulation comprising at least one compound as claimed in claim 16 and at least one solvent and/or at least one further organic or inorganic compound.

28. An organic electroluminescent device comprising at least one compound as claimed in claim 16.

29. An electronic device comprising at least one compound as claimed in claim 16.

30. An organic electroluminescent device which comprises the compound as claimed in claim 16 is used as matrix material and/or in a hole blocker layer and/or in an electron transport layer.
